(19) United States
(12) Patent Application Publication
(10) Pub. No.: US 2012/0029086 A1
(43) Pub. Date: Feb. 2, 2012

(54) Compositions and Method for Temporary Reduction of Nasal and Sinus Pain

(75) Inventor: Richard Jacobsen, Las Vegas, NV (US)

(73) Assignee: Richard Allan Jacobsen, Las Vegas, NV (US)

(21) Appl. No.: 13/280,836

(22) Filed: Oct. 25, 2011

(51) Int. Cl. A61K 31/164; A61P 11/00

(52) U.S. Cl. 514/626

(57) ABSTRACT

Compositions and method that are expedient, safe, non-habit forming, and non-sedating for temporary reduction of nasal and sinus pain comprised of a local anesthetic or local anesthetics in a pharmaceutically acceptable carrier by instillation into the nasal cavity and sinuses by method of nasal spray.

### CROSS-REFERENCE TO RELATED APPLICATIONS

“Not Applicable”.

### STATEMENT REGARDING FEDERALLY SPONSORED RESEARCH OR DEVELOPMENT

“Not Applicable”.

### THE NAMES OF THE PARTIES TO A JOINT RESEARCH AGREEMENT

“Not Applicable”.

### INCORPORATION-BY-REFERENCE OF MATERIAL SUBMITTED ON A COMPACT DISC

“Not Applicable”.

### BACKGROUND OF THE INVENTION

Nasal and sinus pain can have a variety of known and unknown causes including injury, surgery, infection, inflammation, swelling, or contraction of the nasal cavity or sinuses. In cases of severely disabling pain, even 1 minute can seem an eternity. Current treatments include administering narcotics or analgesics, anti-inflammatory drugs, decongestants, or combinations thereof. These can be administered orally, intravenously by injection, or by nasal instillation using nasal drops or nasal spray. While these are effective, the problems are that injections usually require application by a medical doctor, and oral or nasal applications often take at least ten minutes or longer on average to effectively reduce pain. Additionally, the effects may cause drowsiness, sedation impairment, and drug dependency may occur using narcotics or analgesics. These side effects often result in the inability to attend and perform work, or personal tasks. No current treatments or methods provide expedient reduction of pain averaging 5 minutes or less in duration without adverse side effects.

Local anesthetics are drugs used for the interruption of nerve transmission of pain sensations. These drugs prevent perception of pain at the site of administration and many have an expedient effect. Some examples are lidocaine also known as xylocaine and lignocaine (entry 5482, page 950, Merck Index, Fourteenth Edition 2006), benzocaine (entry 1086, page 180, Merck Index, Fourteenth Edition 2006), prilocaine (entry 7743, page 1331, Merck Index, Fourteenth Edition 2006), and procaine also known as novacaine (entry 7757, page 1333, Merck Index, Fourteenth Edition 2006). These examples do not cause drowsiness, sedation impairment, or other adverse side effects of current treatments and methods.

### FIELD OF THE INVENTION

This invention relates to compositions and method that are expedient, safe, non-habit forming, and non-sedating for temporary reduction of nasal and sinus pain.

### DESCRIPTION OF RELATED ART INCLUDING INFORMATION DISCLOSED UNDER 37 CFR 1.97 AND 37 CFR 1.98

U.S. Pat. No. 4,466,973 features the oral application of lidocaine, benzocaine, procaine, and bupivacaine HCl in the treatment of nasal and sinus congestion, dysopthia, and excessive lacrimation. The reference makes no suggestion that the compositions might be used intranasally or to reduce nasal or sinus pain.

U.S. Pat. No. 5,008,289 features the use of capsaicin, lidocaine, and benzocaine in the treatment of allergy and inflammatory nasal disorders and headaches only by specifically mentioned method of nose drops. The anesthetics are included in the invention only to reduce the intense pain from instilling the primary ingredient capsaicin into the nose which is only effective in treating said nasal disorders and headaches over a time period of days. The invention does not mention or suggest use specifically for the reduction of nasal or sinus pain or effectiveness in a short time period of minutes.

U.S. Pat. No. 5,134,166 features the use of capsaicin, lidocaine, and benzocaine in treating nasal congestion, sneezing, or headaches caused by allergy or inflammatory disorders only by the specifically mentioned method of nose drops. The anesthetics are included in the invention only to reduce the intense pain from instilling the primary ingredient capsaicin into the nose which is only effective in treating said nasal disorders and headaches over a time period of days. The invention does not mention or suggest use specifically for the reduction of nasal or sinus pain or effectiveness in a short time period of minutes.

U.S. Pat. No. 6,432,986 features treating cerebral neurovascular disorders and headaches by dorsonasal administration to the sphenopalatine ganglion and mentions the use of ropivacaine, levo-ropivacaine, bupivacaine, levo-bupivacaine and specifically states not using lidocaine as the primary anesthetic. Repeated use of the term “administering to the patient” and “administration over a period of time less than about one half hour” suggests the treatment to only be administered in a clinical environment by a medical doctor suggesting the treatment is not easily self administered or expedient. Use of the invention specifically for the reduction of nasal or sinus pain is not mentioned or suggested.

### BRIEF SUMMARY OF THE INVENTION

It is an object of this invention to provide local anesthetic compositions and method for the temporary reduction of nasal and sinus pain which are portable, expedient, safe, non-habit forming, and non-sedating which currently does not exist. It is yet a further object of this invention to provide a method for the temporary reduction of nasal and sinus pain that is easy to self administer and produces a result in a very short time after application, usually within a few minutes.

### BRIEF DESCRIPTION OF THE SEVERAL VIEWS OF THE DRAWING

“Not Applicable”.

### DETAILED DESCRIPTION OF THE INVENTION

Compositions and method for the temporary reduction of nasal and sinus pain which will not cause drowsiness or impairment compared to current and previous methods, compositions, and treatments. In the practice of this invention a composition is prepared by introducing a safe, expedient, non-habit forming, non-sedating local anesthetic in concentrations of 4.01% or greater into a pharmaceutically acceptable carrier. The resulting composition is used to temporarily reduce nasal and sinus pain by instillation into the nasal cavity and sinuses by administration of an easily self administered nasal spray and repeated as necessary to effectively reduce nasal or sinus pain.

In the best mode or preferred embodiment, a composition is prepared by introducing lidocaine in a concentration of 10% in a pharmaceutically acceptable carrier, and the resulting composition is used to reduce nasal and sinus pain by administration of nasal spray using a easy to use nasal spray applicator such as a single dose or multiple dose applicator. The application is repeated as necessary to effectively reduce nasal or sinus pain.

In another embodiment ambucaine, amethocaine, amolanone, amylocaine, articaine, benoxinate, benzocaine, betoxycaine, biphenamine, bupivacaine, butacaine, butamben, butanilicaine, butethamine, butoxycaine, carbocaine, carticaine, chloroprocaine, cinchocaine, clorprocaine, cocaethylene, cyclomethycaine, dibucaine, dimethisoquin, dimethocaine, diperodon, ecogonidine, ecogonine, eidocaine, etidocaine, eucaine, euprocin, fenalcomine, formocaine, hexylcaine, hydroxyteteracaine, larocaine, leucinocaine, levobupivacaine, levoropivacaine, levoxadrol, lidocaine, lignocaine, marcaine, mepivacaine, meprylcaine, metabutoxycaine, myrtecaine, naepaine, novocaine, octacaine, orthocaine, oxethazaine, parenthoxycaine, phenacaine, piperocaine, piridocaine, polidocanol, pramoxine, prilocaine, procaine, propanocaine, proparacaine, propipocaine, propoxycaine, proxymetacaine, pseudococaine, pyrrocaine, ravocaine, risocaine, ropivacaine, septocaine, stovaine, tetracaine, topicaine, trimecaine, tolycaine, tropane, xylocaine, or zorcaine, or any derivatives thereof and in any combinations thereof comprises the local anesthetic or anesthetics. Selection of optimal doses and formulations are well within the skill of the ordinary artisan skilled in the art.

In another embodiment said composition includes a decongestant or decongestants. The decongestants selected and concentrations thereof can vary depending upon the particular formulation as well as the specific compounds selected. Selection of optimal doses and formulations are well within the skill of the ordinary artisan skilled in the art.

In yet another embodiment said composition includes a buffering agent or agents. The buffers selected and concentrations thereof can vary depending upon the particular formulation as well as the specific compounds selected. Selection of optimal doses and formulations are well within the skill of the ordinary artisan skilled in the art.

In yet another embodiment said composition includes sumatriptan, zolmitriptan, rizatriptan, naratriptan, almotriptan, eletriptan, aspirin, acetaminophen, ibuprofen, naproxen, antihistamines, vasoconstrictors, anti-inflammatory drugs, vitamins, caffeine, flavorings, scents, or odorants or any combination thereof. The drugs or additives selected and concentrations thereof in all embodiments can vary depending upon the particular formulation as well as the specific compounds selected. Selection of optimal doses and formulations are well within the skill of the ordinary artisan skilled in the art.

### Example 1

A 48 year old male suffering from chronic severely disabling episodic nasal and sinus pain each time immediately self administered a nasal spray containing 10% lidocaine in a pharmaceutically acceptable carrier in the affected nasal cavity and sinuses and experienced significant reduction of nasal and sinus pain within an average of 2 minutes and repeated as necessary on recurrence of nasal and sinus pain with similar results. Said subject was then able to attend work and perform work and personal tasks which he could not previously without application of said spray. Previously the subject had to take a schedule 2 orally administered opioid analgesics 1.5 times stronger than morphine to endure the severe pain and the narcotic would average 20 minutes to take effect and did not provide the level of reduction in pain as the spray composition. Each time taking the analgesic said subject could not attend work or perform personal tasks due to the ineffectiveness in reducing pain or adverse side effects.

### Example 2

A 35 year old female suffering from chronic severe sinus pain each time immediately self administered a nasal spray containing 10% lidocaine in a pharmaceutically acceptable carrier in the affected nasal cavity and sinuses and experienced significant reduction of nasal and sinus pain within 5 minutes and repeated as necessary on recurrence of pain with similar results. Said subject was then able to attend work and perform work and personal tasks which she could not previously without application of said spray. Previously the subject had administered non-prescription and prescription oral and nasal treatments, and schedule 3 narcotics orally, with a minimal reduction of nasal and sinus pain. As a result, said subject was not able to attend work or perform personal tasks on the oral and nasal non-prescription and prescription treatments and schedule 3 orally administered narcotics due to the ineffectiveness of reduction of pain or adverse side effects.

### Example 3

A 13 year old male suffering from chronic disabling sinus pain each time immediately self administered a nasal spray containing 10% lidocaine in a pharmaceutically acceptable carrier in the affected nasal cavity and sinuses and experienced significant reduction of nasal and sinus pain within 3 to 5 minutes and repeated as necessary on recurrence of pain with similar results. Said subject was then able to attend school and perform school and personal tasks which he could not previously without application of said spray. Previously the subject had administered non-prescription and prescription oral treatments, and non-prescription nasal treatments with a minimal reduction of nasal and sinus pain. As a result, said subject was not able to attend work or perform personal tasks on non-prescription and prescription oral and non-prescription nasal treatments due to the ineffectiveness of reducing nasal or sinus pain or adverse side affects.

While the foregoing examples presented certain specific embodiments of the invention, it is to be understood that such have been made by way of example only and are not intended to limit the scope of the invention as herein described and claimed.

What I claim as my invention is:

1. A method of temporarily reducing nasal and sinus pain wherein a composition comprising of lidocaine, lidocaine derivatives, benzocaine, benzocaine derivatives, prilocaine, prilocaine derivatives, procaine, or procaine derivatives in concentrations of 4.01% or greater comprises the local anesthetic or combinations thereof in a pharmaceutically acceptable carrier is instilled into the nasal cavity or sinuses and can easily be self administered by instilling said composition into the nasal cavity and sinuses by nasal spray.

2. A method of claim 1 wherein the anesthetic is ambucaine, amethocaine, amolanone, amylocaine, articaine, benoxinate, benzocaine, betoxycaine, biphenamine, bupivacaine, butacaine, butamben, butanilicaine, butethamine, butoxycaine, carbocaine, carticaine, chloroprocaine, cinchocaine, clorprocaine, cocaethylene, cyclomethycaine, dibucaine, dimethisoquin, dimethocaine, diperodon, ecogonidine, ecogonine, eidocaine, etidocaine, eucaine, euprocin, fenalcomine, formocaine, hexylcaine, hydroxyteteracaine, larocaine, leucinocaine, levobupivacaine, levoropivacaine, levoxadrol, lidocaine, lignocaine, marcaine, mepivacaine, meprylcaine, metabutoxycaine, myrtecaine, naepaine, novocaine, octacaine, orthocaine, oxethazaine, parenthoxycaine, phenacaine, piperocaine, piridocaine, polidocanol, pramoxine, prilocaine, procaine, propanocaine, proparacaine, propipocaine, propoxycaine, proxymetacaine, pseudococaine, pyrrocaine, ravocaine, risocaine, ropivacaine, septocaine, stovaine, tetracaine, topicaine, trimecaine, tolycaine, tropane, xylocaine, or zorcaine, or any derivatives thereof or any combinations thereof.

3. A method of claim 1 further including in the composition sumatriptan, zolmitriptan, rizatriptan, naratriptan, almotriptan, eletriptan, aspirin, acetaminophen, ibuprofen, naproxen, antihistamines, vasoconstrictors, anti-inflammatory drugs, decongestants, buffering agents, vitamins, caffeine, flavorings, scents, or odorants or any combinations thereof.

4. A method of claim 1 wherein repeating application a sufficient number of times to affect the temporary reduction of nasal and sinus pain.
